(19) United States
(12) Patent Application Publication
(10) Pub. No.: US 2004/0215194 A1
(43) Pub. Date: Oct. 28, 2004

(54) Osteosynthetic device

(76) Inventors: Markus Hehli, Davos Frauenkirch (CH); Alberto Fernandez Dell'Oca, Montevideo (UY)

Correspondence Address: JONES DAY, 51 Louisiana Aveue, N.W, WASHINGTON, DC, 20001-2113, US

(21) Appl. No.: 10/694,846

(22) Filed: Oct. 29, 2003

Related U.S. Application Data
(63) Continuation of application No. PCT/CH01/00276, filed on May 3, 2001.

(30) Foreign Application Priority Data
May 3, 2001 (WO) PCT/CH01/00276

(51) Int. Cl. A61B 17/58

(52) U.S. Cl. 606/63

(57) ABSTRACT

The invention relates to an osteosynthetic device (1) in form of an intramedullary nail with a longitudinal shape, a central axis (5), a first end (2) and a second end (3). The device (1) is helically shaped.

### CROSS-REFERENCE TO RELATED APPLICATION

This application is a continuation of the U.S. National Stage Designation of co-pending International Patent Application PCT/CH01/00276, filed May 3, 2001. The entire content of this application is expressly incorporated herein by reference thereto.

### FIELD OF THE INVENTION

The invention relates generally to a helically shaped intramedullary nail capable of following the shape of the intramedullary canal of long bones of humans.

### BACKGROUND OF INVENTION

The intramedullary canal of a long bone must be prepared for insertion of an intramedullary nail. Prior art intramedullary nails have the following major disadvantages:

The opening is bigger than the nail cross-section because of the nail; and

The single bend of at one point of the nail does not correspond to the anatomical shape of the intramedullary canal of long bones.

Conventional nails force the surgeon to use a medial entry point in the proximal humerus which is located near the articular surface of the humerus, i.e., far from ideal from the mechanical and vascular standpoint.

### SUMMARY OF INVENTION

The present invention is designed to overcome the foregoing problems by providing an osteosynthetic device, particularly an intramedullary nail, which is capable of following the shape of the intramedullary cavity of long bones of humans. No oversized opening is needed because the helix shape makes it possible to turn the nail during its insertion into the intramedullary cavity. The entry point of unreamed nails is optimized especially at the femur and the tibia, but also in the humerus.

The main advantages of the device according to the invention are the following:

It allows for a better placement of the entry hole of the nail into the bone avoiding problems such as injuring the vascular supply of the femoral head, thus lowering the risk of complications and facilitating humeral nailing;

It does not require an entry hole larger than the cross-section of the nail; and

It allows for easy removal of the nail after the bone has healed.

Elastic intramedullary nails are not so useful in adolescent children because they may be slightly unstable, often requiring the use of postoperative splints. The use of conventional nails in older children and adolescents is associated with a high risk of femoral head necrosis. Lateral entry points of a thin constant cross-section can be advantageous for these patients.

With plates and/or internal fixators according to the present invention, the main advantage is the option of allowing the implant to be, e.g., anterior in the distal humerus and lateral in the proximal humerus, avoiding the risk of radial nerve injury.

While one of the principal applications of the invention is as an intramedullary nail, the invention can also be applied to extramedullary devices, e.g., bone plates or internal fixators.

The intramedullary nail according to the present invention may be used in the femur, humerus, tibia, and radius.

In a preferred embodiment of the present invention, the envelope of the helix is a cylinder having the same central axis as the helix and the helix has a total rotation of less than 540°, preferably less than 360°. The radius of the cylinder is in the range of 10 to 50 mm, preferably in the range of 15 to 30 mm. The pitch of the helix should be in the range of 100 to 1500 mm, preferably in the range of 300 to 1000 mm. The cross-section orthogonal to the central axis of the helix is preferably a circle, square or star.

In another preferred embodiment, the second end of the nail is pointed, which facilitates introduction into the bone.

In another preferred embodiment, the cross-section orthogonal to the central axis of the helix is essentially a rectangle with sides a and b, the larger sides b being oriented to the outer and inner side of the helix. The ratio of a:b is smaller than 0.5, preferably smaller than 0.35. Preferably, the essentially rectangular cross-section is rounded at its smaller sides a.

In another preferred embodiment, the portion of the helix near the first end is thicker than the portion of the helix near the second end. This allows for attachment of a handle to hold and manipulate the helical nail.

In another preferred embodiment, the central axis of the helix is a straight line.

In another preferred embodiment, the cross-section orthogonal to the central axis has a maximum dimension in the range of 5 to 14 mm and the length of the cylinder of the helix is in the range of 200 to 500 mm.

In another preferred embodiment, the implant may be provided with lateral holes for locking screws.

### BRIEF DESCRIPTION OF THE DRAWINGS

The invention will be further described with reference to the accompanying drawings, wherein:

FIG. 1 is a perspective view of a device according to the present invention in the form of a helical nail;

FIG. 2 is a perspective view of a device according to the invention in the form of a helical plate;

FIG. 3 is a detail of the nail according to FIG. 1;

FIG. 4 is a detail of the plate according to FIG. 2;

FIG. 5 is an orthogonal cross-section through the nail according to FIG. 1;

FIG. 6 is a variation of the orthogonal cross-section; and

FIG. 7 is a further variation of the orthogonal cross-section.

### DETAILED DESCRIPTION OF THE PREFERRED EMBODIMENTS

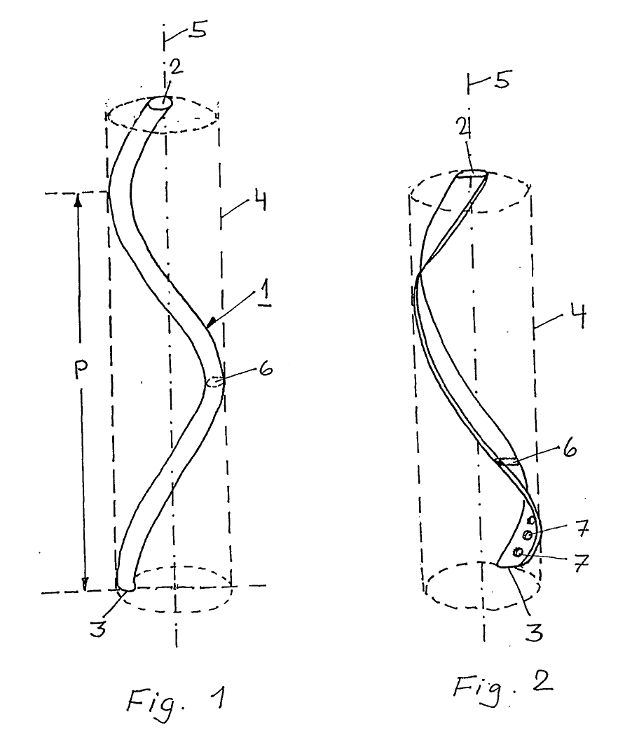

The osteosynthetic device 1 according to the invention is represented in FIG. 1 in the form of an intramedullary nail. Intramedullary nail 1 has a longitudinal shape with a central axis 5, a first end 2, and a second end 3. The shape of intramedullary nail 1 is a helix, with the envelope of the helix being a cylinder 4 having the same central axis as the helix. The central axis 5 of the helix is a straight line. The helix has a rotation of less than 540°, preferably less than 360°. Typically, the helix has a rotation of over 240°. The radius r of the cylinder 4 is in the range of 10 to 50 mm, preferably in the range of 15 to 30 mm. The pitch p of the helix is in the range of 100 to 1500 mm, preferably in the range of 300 to 1000 mm. As shown in FIG. 5, the cross-section orthogonal to the central axis 5 of the helix is a circle, i.e., the helix is made of a cylindrical rod. Alternatively, as shown in FIGS. 6 and 7, the cross-section may also have the shape of a square or a star, or may be fluted.

Another embodiment of the invention is represented in FIG. 2. It differs from the embodiment of FIG. 1 in that the cross-section orthogonal to the central axis 5 is rectangular, i.e., the helix is made of a flattened rod. In particular, the cross-section 6 orthogonal to the central axis 5 of the helix is essentially a rectangle with the sides a and b, the larger side b being oriented to the outer and inner side of the helix. Instead of a rectangular shape, the cross-section could have an ellipsoidal shape, where a/2 and b/2 would be the half-axes of the ellipse. The ratio of a:b should be smaller than 0.5, preferably smaller than 0.35.

The portion of the helix near the first end 2 is thicker than the portion of the helix near the second end 3, allowing attachment of a handle to hold and manipulate the intramedullary nail 1. The cross-section orthogonal to the central axis 5 has a maximum dimension in the range of 5 to 14 mm.

As shown in FIG. 3, the second end 3 of the intramedullary nail 1 is pointed for easier introduction into the bone.

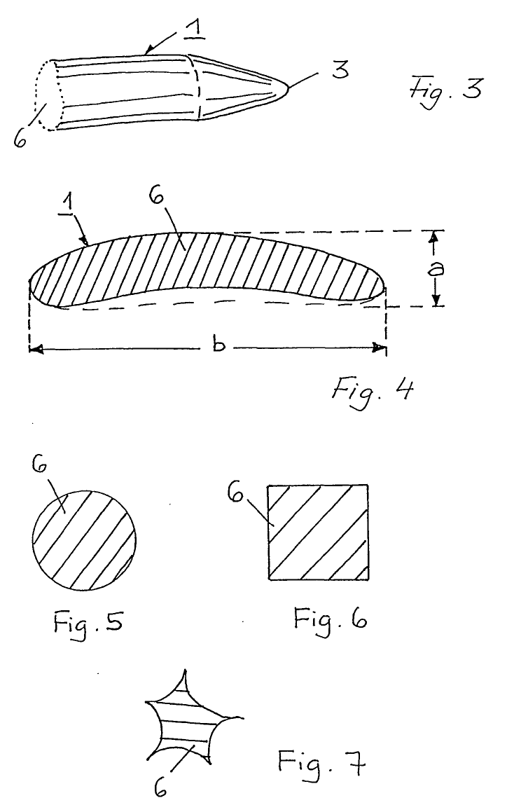

As shown in FIG. 4, the essentially rectangular cross-section of the intramedullary nail 1 is trimmed at its smaller sides a.

FIGS. 5-7 show different cross-sections of the nail according to the invention.

The devices according to this invention may be made of any appropriate material, depending on the purpose to be served. The devices may be made of metals, e.g., an appropriate alloy of stainless steel or titanium, pure titanium, or a polymeric material (including composites).

1. An osteosynthetic device, in particular an intramedullary nail, having a longitudinal shape with a central axis, a first end, and a second end, wherein the shape of the device is helical.

2. The osteosynthetic device of claim 1, wherein the envelope of the helix is a cylinder having the same central axis as the helix.

3. The osteosynthetic device of claim 1, wherein the helix has a rotation of less than 540°, preferably less than 360°.

4. The osteosynthetic device of claim 1, wherein the radius of the cylinder is in the range of 10 to 50 mm, preferably in the range of 15 to 30 mm.

5. The osteosynthetic device of claim 1, wherein the pitch of the helix is in the range of 100 to 1500 mm, preferably in the range of 300 to 1000 mm.

6. The osteosynthetic device of claim 1, wherein the pitch of the helix is greater than 400 mm, preferably larger than 600 mm.

7. The osteosynthetic device of claim 1, wherein the cross-section orthogonal to the central axis of the helix is a circle.

8. The osteosynthetic device of claim 1, wherein the cross-section orthogonal to the central axis of the helix is a square or a star.

9. The osteosynthetic device of claim 1, wherein the second end is pointed.

10. The osteosynthetic device of claim 1, wherein the cross-section orthogonal to the central axis of the helix is essentially a rectangle with the sides a and b, the larger side b being oriented to the outer and inner sides of the helix.

11. The osteosynthetic device of claim 10, wherein the ratio of a:b is smaller than 0.5, preferably smaller than 0.35.

12. The osteosynthetic device of claim 10, wherein the essentially rectangular cross-section is rounded at its smaller sides a.

13. The osteosynthetic device of claim 1, wherein the portion of the helix near the first end is thicker than the portion of the helix near the second end.

14. The osteosynthetic device of claim 1, wherein the central axis of the helix is a straight line.

15. The osteosynthetic device of claim 1, wherein the cross-section orthogonal to the central axis has a maximum dimension in the range of 5 to 14 mm, preferably in the range of 7 to 11 mm.

16. The osteosynthetic device of claim 1, wherein the length of the cylinder or of the helix is in the range of 200 to 500 mm, preferably in the range of 250 mm to 400 mm.

17. The osteosynthetic device of claim 1, wherein the device is provided with through holes for locking screws, preferably near the second end.

18. The osteosynthetic device of claim 1, wherein the device is provides with at least two, preferably with at least three through holes for locking screws.
